(19) United States
(12) Patent Application Publication
(10) Pub. No.: US 2008/0248478 A1
(43) Pub. Date: Oct. 9, 2008

(54) MOLECULAR HISTOLOGICAL ANALYSIS OF MULTICELLULAR SAMPLES

(76) Inventors: Jochen Renzing, Iffeldorf (DE); Roderic Fuerst, Village Neuf (FR); Martin Gutekunst, Eberfing (DE)

Correspondence Address: ROCHE DIAGNOSTICS OPERATIONS INC., 9115 Hague Road, Indianapolis, IN, 46250-0457, US

(21) Appl. No.: 12/020,619

(22) Filed: Jan. 28, 2008

(30) Foreign Application Priority Data
Feb. 2, 2007 (EP) 07002338.7
Mar. 6, 2007 (EP) 07103577.8

(51) Int. Cl. C12Q 1/68; C12M 1/00; G01N 33/53; G06K 9/78; C12Q 1/02

(52) U.S. Cl. 435/6; 435/288.7; 435/287.2; 435/29; 435/7.2; 382/133

(57) ABSTRACT

The present invention provides modified digital images of a multicellular sample using molecular profiles of cells from said multicellular sample for a molecular histological analysis.

### RELATED APPLICATIONS

This application claims priority to European application EP 07103577.8 filed Mar. 6, 2007.

### FIELD OF THE INVENTION

The present invention provides modified digital images of a multicellular sample using molecular profiles of cells from said multicellular sample for a molecular histological analysis. It provides a new level of data quality for an improved diagnostic output of the examination of histopathological samples by providing a virtual digital image of predictive or diagnostic value.

### BACKGROUND OF THE INVENTION

Recent technological advances allow the precise measurement of single-cell molecular profiles. For instance, Bengtson, M. et al., Genome Research 15:1388-1392, 2005, have studied the expression of multiple genes in individual mouse pancreatic islet cells by reverse transcriptase quantitative real-time PCR (RT-QPCR). This technique affords superior sensitivity, accuracy, and dynamic range compared with that of alternative methods for gene expression analysis. Schlieben et al. have previously described methods for the mRNA isolation from individual limited cell samples and single cells (Technical Note TN41000-003, Bio-Nobile Oy, 2004). In an attempt to enhance the processing of morphological data derived from microscopic imaging, Kotsianti et al. have described a workflow from image acquisition of morphological data to pathological classification of tissue (US 2005/0165290). Further, there are descriptions available of how a combination of biomarker data and bio-image data may be used to derive a predictive diagnosis (Saidi et al., US 2005/0108753).

Options to isolate small numbers of purified cells from complex cellular samples such as micromanipulation, fluorescence-activated cell sorting, or laser microdissection are, e.g., described in the following review articles: Burgemeister, R., J. Histochem. Cytochem. 53:409-412, 2005, and Baech, J. and Johnsen, H. E., Stem Cells 18:76-86, 2000.

However, presently there are no seamless data processing solutions available to combine the morphological information derived from microscopic imaging of biological samples with subsequently derived information on the molecular state of the processed samples, in particular, molecular data derived on the single cell level.

The disparity between the ability to measure and the desire to make biological or diagnostic conclusions calls out for a new approach for data processing and data handling in this context.

In general, a pathologist or researcher uses a visual assessment for the determination of disease status or morphological Status of a cell-containing sample. Therefore, in order to enhance cell analysis and speed up sample throughput, a computer aided system for assessment and diagnosis is desirable, enabling an automation of the analysis process that converts digital images and primary molecular data into summary data for use by a researcher or pathologist. In other words, a method is needed that integrates molecular data obtained by cell analysis with morphological data of cell images in one seamless process.

### SUMMARY OF THE INVENTION

The present invention provides a new level of data quality for an improved diagnostic output of the examination of histopathological samples by providing a virtual digital image of predictive or diagnostic value.

To solve the above mentioned problem, this invention provides a method, a computer program, a database and a system to improve data processing and data handling for molecular histological analysis of a multicellular sample to gain a new level of data quality by retaining the contextual morphological information of measured molecular cell values.

In other words, the results of measurements performed with cells from a multicellular sample are combined with morphological information contained in digital images. The results should be integrated into the image in such a way that a good impression of the morphology and histological status of the cells is retained for examination by the user. This integration allows the visualization of cellular quantities in the location of individual cells and therefore, direct comparison of cells of the multicellular sample.

One aspect of the present invention is a system to perform a molecular histological analysis of a multicellular sample, the system comprising:
an optical device to produce a digital image of said multicellular sample,
a computer means to select a region of interest within said digital image corresponding to one cell or a number of cells within said multicellular sample,
an isolation device to isolate said cell or said number of cells selected by the computer means from said multicellular sample,
an analysis device to perform a biochemical analysis and to produce a molecular profile of said isolated cell or said number of isolated cells, said molecular profile comprising a plurality of molecular values,
a software application to store said digital image and said molecular profile in such a way that said molecular profile is linked to the corresponding selected region of interest within said digital image, and
a visualizing means to present a modified digital image comprising said digital image produced by the optical device together with one or more of said molecular values produced by the analysis device, wherein said molecular histological analysis is based on said modified digital image.

Throughout the present description of the invention, a molecular histological analysis is the analysis of an multicellular sample with respect to morphological as well as molecular aspects. A multicellular sample is an assembly of cells, such as in a tissue sample or a cell culture.

In order to gain the morphological information of the multicellular sample, the system of the present invention has an optical device to record a digital image of the multicellular sample. As optical device, all kinds of devices known to someone skilled in the art are suitable, such as devices to perform light microscopy, contrast microscopy, confocal microscopy, or fluorescence microscopy.

Within the recorded digital image, the system selects cells that are of further interest and defines a region within said digital image corresponding to the cell of interest. This region within the recorded digital image is called region of interest (ROI) throughout the present description. This ROT is designed by the computer means as the exact contour of the cell having a complex shape or as a simple shape (like a circle or a square) comprising the cell and part of its surrounding. Alternatively, such an ROI can also be defined to include more than one cell.

The selection of the ROI can be initiated by manual input by the user indicating the cells of interest or automatically by the computer means using a pattern recognition algorithm. Using such a pattern recognition algorithm, it is possible that the computer means not only selects the ROI but also identifies the cells of interest.

The selected ROIs are than used by the isolation device to isolate said cell or said number of cells from said multicellular sample that corresponds to the selected regions within the digital image. The phrase “number of cells” is used throughout the present invention to emphasize that it is not necessary for all applications of the present system to isolate a plurality of single cells from the multicellular sample, but that groups of neighboring cells can be sufficient. Afterwards, such a group of neighboring cells from the multicellular sample is further processed together in order to produce a single molecular profile.

Subsequently, the isolated cell or cells of each ROI are placed in a separate storage device for further processing. Said storage device can be, e.g., a separate vessel for each ROI or a multiwell plate wherein each well contains the cell or cells from one ROI.

The analysis device is necessary to perform a biochemical analysis with the isolated cells in order to produce molecular profiles. For this purpose, the analysis device of the present invention is able to perform a separate biochemical analysis for each storage device containing cells from one ROI.

The analysis device produces molecular profiles of cells isolated from said multicellular sample. This implies that a lysis of the cells is required to reach the molecular constituents such as nucleic acids and proteins. The phrase “molecular profile” is used throughout the present description of the invention to emphasize that more than one molecular constituent of the respective cells is analyzed. Therefore, throughout the present description of the invention, a molecular profile comprises a plurality of molecular values obtained by isolating a cell or a number of cells from a multicellular sample. Such molecular values of the corresponding molecular constituent can be, e.g., the concentration, the absolute amount, a relative ratio with respect to a control or reference, or a physical quantity as qualitative measure.

The gained digital image and molecular profile are processed by a software application. Throughout the description of the present invention, said software application must enable that the digital images are stored together with the molecular profile in such a way that each molecular profile is linked to the selected region of interest within said digital image corresponding to the cells that were isolated to obtain said molecular profile. In general, there are at least two possible alternatives to provide such a software application, namely a data-based application or an image-based application.

Finally, the system according to the present invention comprises a visualizing means to present modified digital images to the user. A modified digital image is based on the original digital image recorded by the optical device that is modified with a certain number of molecular values in such a way that said molecular values are visualized within the digital image and that each molecular value is clearly linked to the region of interest within the modified digital image corresponding to its cellular origin.

Another aspect of the present invention is a method to perform a molecular histological analysis of a multicellular sample comprising:
a) obtaining a digital image of said multicellular sample,
b) selecting a region of interest within said digital image corresponding to one cell or a number of cells within said multicellular sample,
c) isolating said cell or said number of cells selected in step b) from said multicellular sample,
d) performing a biochemical analysis to produce a molecular profile of said isolated cell or said number of isolated cells, said molecular profile comprising a plurality of molecular values, and
e) producing a modified digital image comprising said digital image obtained in step a) together with one or more of said molecular values produced in step d) to perform said molecular histological analysis.

Yet another aspect of the present invention is a method to identify the location of characteristic cells within a multicellular sample in order to perform a molecular histological analysis, said method comprising:
a) providing a digital image of said multicellular sample together with a molecular profile of a cell or number of cells within said multicellular sample, each molecular profile comprising a plurality of molecular values and linked to a region of interest within said digital image corresponding to the respective cell or number of cells,
b) highlighting in a modified digital image those cells within said multicellular sample that fulfill a defined molecular criteria,
   wherein said molecular profiles are obtained by isolating a cell or a number of cells from said multicellular sample.

This embodiment of the present invention refers to a method that enables a user to identify those cells within a multicellular sample that fulfill a defined molecular criteria by examination of a modified digital image. Cells that fulfill such a molecular criteria are referred to as “characteristic cells” throughout the present description of the invention, and identification of said characteristic cells is necessary to perform the molecular histological analysis of the multicellular sample.

There are many different molecular criteria that are of interest for a molecular histological analysis of a multicellular sample. For example, all cells within the multicellular sample can be highlighted that exceed a certain threshold with respect to the concentration of a cellular constituent. It can also desirable to visualize more complex molecular criteria such as a concentration relative to a reference or control, a concentration criteria combined with a distance demand or a molecular amount combined with a morphological criteria.

Using the method to identify the location of characteristic cells according to the present invention, a plurality of histological hypotheses can be tested based on a digital image of a multicellular sample and a certain number of molecular profiles of cells of this multicellular sample.

Still another aspect of the present invention is a computer program to generate a plurality of modified digital images from the digital image of a multicellular sample, said digital images being modified with one or more molecular values of a respective cell or number of cells within said multicellular sample, said computer program comprising:
means for loading said digital image of said multicellular sample and a plurality of molecular values of a cell or number of cells from said multicellular sample, each molecular value being linked to a region of interest within said digital image corresponding to the respective cell or number of cells, and
means for generating a modified digital image comprising said digital image and a number of selected molecular values from said plurality of molecular values, wherein said molecular values are obtained by isolating a cell or a number of cells from said multicellular sample.

Such a computer program generates a plurality of modified digital images in order to perform, e.g., a molecular histological analysis that is based on said modified digital image.

This computer program according to the present invention enables a user to generate modified digital images that highlight those cells within a multicellular sample that fulfill a certain user-defined molecular criteria. This modified digital image can be used, e.g., to verify histological hypotheses.

Moreover, an aspect of the present invention is a method for managing molecular histological experiments of a multicellular sample, said method comprising:
a) loading a digital image of said multicellular sample into a software application,
b) defining regions of interest within said digital image that are linked to corresponding positions of cells within said multicellular sample, and
c) loading a plurality of molecular profiles of cells or number of cells from said multicellular sample into said software application, each molecular profile comprising a plurality of molecular values, wherein said software application is structured such that said plurality of molecular profiles loaded in step c) are linked to the corresponding region of interest defined in step b) within said digital image loaded in step a), and wherein said molecular profiles are obtained by isolating a cell or a number of cells from said multicellular sample.

In addition, the present invention relates to a computer program to identify the location of characteristic cells within a multicellular sample in order to perform a molecular histological analysis, said computer program comprising:
a first application to load a digital image of multicellular sample, to define regions of interest within said digital image that are linked to corresponding positions of cells within said multicellular sample, and to save said digital image together with said regions of interest as a first image file,
a second application to combine said first image file with a plurality of molecular profiles comprising a plurality of molecular values and to save said first image file together with said plurality of molecular profiles as a second image file, wherein said plurality of molecular profiles are linked to the corresponding region of interest within said digital image, and
a third application to extract modified digital images from said second image file according to user-defined molecular criteria, wherein said modified digital images highlight cells that fulfill said user-defined molecular criteria, wherein said molecular profiles are obtained by isolating a cell or a number of cells from said multicellular sample.

The computer program according to this embodiment of the present invention comprises three different applications performing parts of the overall computer program.

The first application is an imaging application to load a digital image and to define ROIs within said digital image. As mentioned before, the ROI can be the exact contour of the cell having a complex shape or a simple shape (like a circle or square) comprising the cell and part of its surrounding. Alternatively, such an ROI can include more than one cell. The definition of the ROIs can be initiated manually by input by the user of the system indicating the cells of interest or automatically by a pattern recognition algorithm. For the alternative of manual input, the first application requires the possibility to display the digital image for examination by the user. Moreover, said first application has a saving function to save the digital image together with the defined ROIs, wherein the file comprising the digital image and the defined ROIs is called a first image file.

The second application is able to add to said first image file a plurality of molecular profiles and to save both components together as a second image file. The second application is designed to link the molecular profiles to the corresponding ROIs representing the cells of the multicellular sample that provided said molecular profiles.

The third application is necessary to extract modified digital images from the second image file. As mentioned before, a modified digital image is based on the original digital image modified with a certain number of molecular values, whereas each modified digital image is created according to a user-defined criteria. Different kinds of criteria were mentioned before and comprise, e.g., cells with a concentration of a cellular constituent above a certain threshold or cells with a certain relative concentration.

The present invention also relates to a database for managing molecular histological experiments of a multicellular sample, said database comprising:
a cell table comprising cell numbers together with the corresponding position within said multicellular sample,
a molecular value table comprising molecular profiles of cells within said multicellular sample, each molecular profile comprising a plurality of molecular values, and
a digital image table comprising digital images of said multicellular sample and coordinates of cells within said digital image,
   wherein said cell table is linked with said molecular value table and with said digital image table such that each cell of said multicellular sample is linked to the corresponding coordinates within the respective digital image and to the corresponding molecular profile,
   wherein said database is designed such that a modified digital image based on a digital image from said digital image table highlighting cells fulfilling a plurality of user-defined molecular criteria is producible, and
   wherein said molecular profiles are obtained by isolating a cell or a number of cells from said multicellular sample.

The database according to this embodiment of the present invention comprises at least three substructures, namely a cell table, a molecular value table, and a digital image table that are linked to each other in order to produce modified digital images based on a digital image from said digital image table and a certain number of molecular values from said molecular value table. This database is structured such that a plurality of modified digital images can be extracted upon defining a certain molecular criteria.

The cell table summarizes information about each cell of interest that is used for the molecular histological experiment, namely the position of said cell within the multicellular sample together with an assigned identification number and, if necessary, an analysis number (e.g., a well number) for the subsequent biochemical analysis.

The molecular value table comprises all molecular values obtained by the biochemical analysis, whereas all molecular values belonging to a respective cell are grouped as a molecular profile.

In the digital image table, the digital images of the multicellular sample are stored together with coordinates that define the ROI corresponding to the cells of interest within the respective digital image.

### BRIEF DESCRIPTION OF THE DRAWINGS

FIG. 1 depicts a flow chart of the general method according to the present invention.

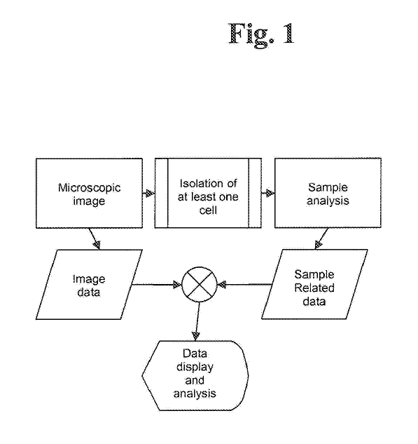

FIG. 2 shows a detailed flow chart of the method according to the present invention.

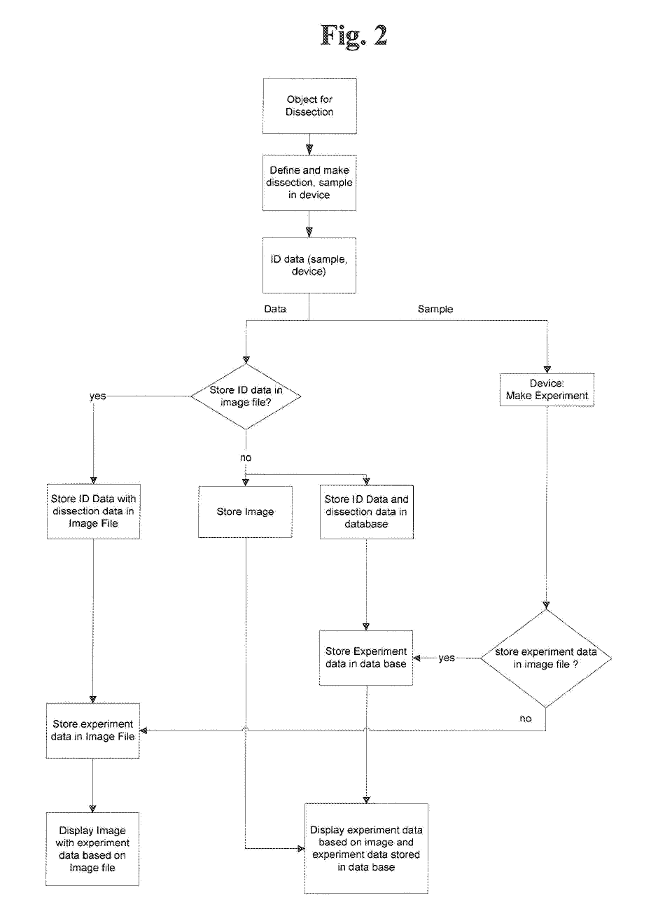

FIG. 3 shows a flow chart of one embodiment of the present invention.

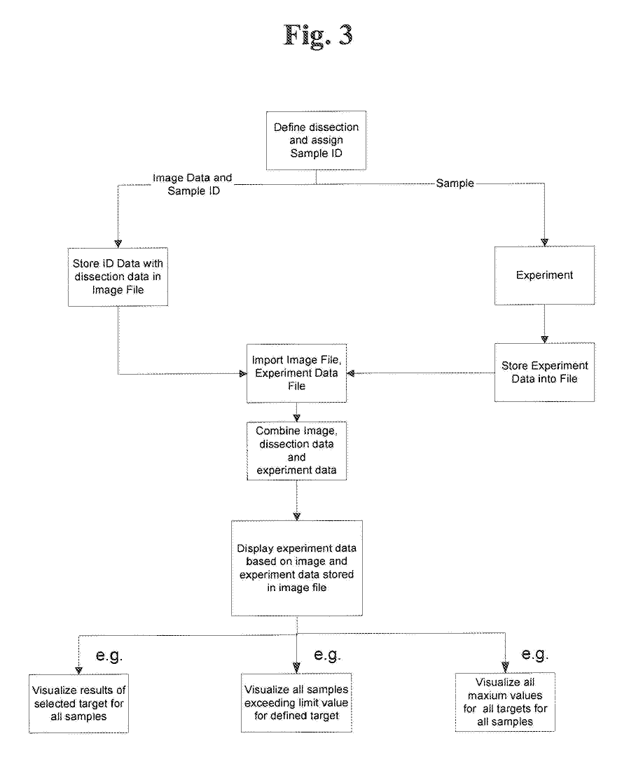

### DETAILED DESCRIPTION OF THE INVENTION

One aspect of the present invention is a system to perform a molecular histological analysis of a multicellular sample comprising:
an optical device to produce a digital image of said multicellular sample,
a computer means to select a region of interest within said digital image corresponding to one cell or a number of cells within said multicellular sample,
an isolation device to isolate said cell or said number of cells selected from said multicellular sample,
an analysis device to perform a biochemical analysis and to produce a molecular profile of said isolated cell or said number of isolated cells, said molecular profile comprises a plurality of molecular values,
a software application to store said digital image and said molecular profile in such a way that said molecular profile is linked to the corresponding selected region of interest within said digital image, and
a visualizing means to present a modified digital image comprising said digital image together with one or more of said molecular values, wherein said molecular histological analysis is based on said modified digital image.

An aspect of the system according to the present invention is the selection of the region of interest (ROI) by the computer means in selecting an ROI. As mentioned before, there are different alternatives to define ROIs within the digital image of the optical device, e.g., to define the ROI as the contour of the cell of interest or as a circle or square spanning the expanse of the cell of interest.

In a preferred embodiment of the present invention, said region of interest is defined by two or more coordinates.

The simplest way to define an ROI requires only two coordinates defining the center of a circle spanning the expanse of the cell of interest. On the other hand, an ROI expressing the contour of the cell requires a large number of coordinates.

In another preferred embodiment of the present invention, said optical device is disposed to perform light microscopy.

As mentioned before a plurality of different optical devices that are known to someone skilled in the art are suitable for the present invention. Examples for devices to perform light microscopy are a commercial light microscope, a contrast microscope, and a confocal microscope. Using light microscopy to produce digital images of the multicellular sample may result in the necessity to treat the sample with compounds that enhance the optical contrast. Such contrast enhancers are known to someone skilled in the art. The most widely used staining technique is that of haematoxylin and eosin, commonly called H&E. In this method the nuclei of cells are stained by the haematoxylin while the cytoplasm is colored by the eosin. This technique in various forms has been around for over one hundred years. Waldeyer was the first person to try staining histological section with an extract of logwood in 1863 (Waldeyer, W., Zeitschr. f. rat. Med. (Dritte R.) Bd. XX (1863) 193-256). The stain haematoxylin is not a dye but develops coloring properties on oxidation to haematin. This substance haematin has little or no affinity for tissue elements and requires an inorganic ion to act as a “go between” (called a mordant) between the dye and the tissue. The mordant may be incorporated into the haematoxylin dye bath, which is the most common method. Or one may pre-treat the tissue with the metal salt and then stain by the haematoxylin. Eosin is an acid dye which requires an acidic environment to work. In solution the dye molecule is negatively charged and thus attaches to positive sites in the tissue by salt bridges.

Moreover, it is of course necessary to use an optical device that provides a magnification of the cellular structures.

In yet another preferred embodiment of the present invention, said optical device employs fluorescence optics, preferably a fluorescence microscope.

Using fluorescence microscopy provides advantages compared to light microscopy such as resolution, multiplexing, and contrast. Moreover, established analysis techniques for tissue and cell cultures such as FISH (fluorescence in-situ hybridization) or fluorescent labeled antibodies, for tissue (immuno histochemistry) or cell cultures (immuno cytochemistry), can be used to gain some molecular information already during the step of image recording.

In an also preferred embodiment of the present invention, said isolation device is a microdissection device, preferably a laser assisted microdissection device.

In yet another preferred embodiment of the present invention, said isolation device is a manual microdissection device, preferably a micropipetting device.

Devices and methods that are suitable for the present invention to isolate single cells from cell cultures or tissue samples are known to someone skilled in the art. Options to isolate small numbers of purified cells from complex cellular samples such as micromanipulation, fluorescence-activated cell sorting, or laser microdissection are, e.g., described in the review article of Player, A., et al., Expert. Rev. Mol. Diagn. 4:831-840, 2004. Laser microdissection devices are commercially available, e.g., from PALM Microlaser Technologies GmbH, Bernried, Germany.

A preferred system according to the present invention is a system, wherein said number of isolated cells comprises less than 100, preferably less than 10, most preferably less than 5 cells.

Depending on the application of the system and the properties of the used analysis device, different requirements with respect to the number of cells per isolation can apply. As mentioned before, a number of cells that are isolated in a single isolation step are always a group of neighboring cells from the same part of the multicellular sample. In general, it is of course preferred to isolate as few cells as possible, ideally just one cell in order to gain a maximum of information about the multicellular sample. There are commercially available microdissection devices able to isolate single cells from tissue, e.g., from PALM Microlaser Technologies GmbH (Bernried, Germany).

Another preferred system according to the present invention is a system wherein said analysis device is capable of performing a downstream analysis process, preferably said analysis device is an immunoassay device, a mass spectrometer, a nucleic acid sequencing device, a nucleic acid hybridization assay device, a surface plasmon resonance device, or gel electrophoresis device. More preferably said analysis device is a PCR device, and most preferably said analysis device is a real-time PCR device.

Throughout the present invention the phrase “downstream analysis process” summarizes all processes that are suitable to gain further information about the isolated cells on a molecular level. Within the present invention, said further information on a molecular level is obtained after isolation of the cells in an external biochemical analysis. But note that this does not exclude the possibility to gain additional molecular cell information already during image recording based on labeled antibodies or in-situ hybridization.

From the group of suitable downstream analysis processes to gain further molecular information on basis of nucleic acids, a PCR device is especially preferred because of the small amount of nucleic acid that in general is obtained from only a number of isolated cells. Moreover, using a real-time PCR provides the desired amplification and the required analysis in a single process. Additionally, real-time PCR offers multiplexing capabilities that are helpful to gain a molecular profile of the cells. One biochemical analysis to which this invention can be applied is the quantification of mRNA derived from individual cells isolated from a multicellular sample.

Yet another preferred system according to the present invention is a system wherein said software application is capable of storing a plurality of molecular profiles, each linked to the corresponding selected region of interest within said digital image.

Since the system of the present invention is applicable for the molecular histological analysis of a multicellular sample, it is necessary that the software application is able to handle multiple molecular profiles that are linked to different regions within a single digital image.

In general, there are at least two alternatives to store a digital image together with multiple molecular profiles and their allocation to respective regions of interest, namely a database or an image file.

In a preferred embodiment of the present invention, said software application is a database, an image-based application, or a combination thereof.

In another preferred embodiment of the present invention, said image-based application is a TIFF application.

The TIFF format allows storing additive information and image overlays by block structure as well as headers describing the block content. To use the TIFF format for the present invention, a software module has to be used to read the TIFF format and to display the image with a data overlay.

The software application annotates an object in the image by an identifier which correlates to the position of the object and/or its morphological structure and should also include the name or number of the respective image. This identifier is also linked to molecular profiles of the object corresponding to said identifier that are stored in said block structure or, e.g., in a relational database.

Finally, the software application can display the image together with the molecular profiles of the objects. This can iteratively be done by the user marking objects defined by the position and/or the morphological structure that correspond to an identifier (including the image name or image number). The software can now display the molecular profile of the object within the image by reading data corresponding to the identifier from the database or the block structure. This workflow also works for displaying information of several objects in parallel.

In a preferred embodiment of the present invention, said visualizing means presents digital images together with molecular values from a plurality of selected regions of interest within said digital image.

As mentioned before, the system of the present invention is applicable for the molecular histological analysis of a multicellular sample, and therefore, it is necessary that the visualizing means can handle a plurality of molecular values belonging to different regions of interest.

In another preferred embodiment of the present invention, said visualizing means presents digital images together with molecular values chosen by a user.

In order to indicate the required molecular values within the modified digital image, several alternatives exist. First of all, a molecular value can be displayed as a numerical value at the position of the respective cell, said numerical value can be, e.g., the concentration of said molecular compound. Alternatively, the molecular value can be displayed by color, e.g., in such a way that a concentration is displayed at the position of the respective cell according to a color scale ranging from white to black or from white to red. Using different color scales a certain number of different concentrations can be displayed simultaneously. Moreover, single colors can be used to mark those cells that fulfill certain molecular criteria, e.g., all cells that have protein A and protein B are marked red. Also the intensity of a single color can be used to display molecular values.

With this kind of visualization, the present invention is suitably applied to a plurality of user-defined processes, e.g., the process of measuring populations of cells that fulfill different molecular criteria based on molecular species that are derived from individual cells contained in biological samples.

In an also preferred embodiment of the present invention, said multicellular sample is a tissue sample, a cell culture sample, a biopsy sample, or a cytological sample.

Such tissue samples or cell culture samples are typically used for research applications. A biopsy sample such as a histopathological sample or a fine needle aspirate are also used for medical or diagnostic applications. For other medical or diagnostic applications of the system of the present invention, said multicellular sample can be a cytological sample such as a blood smear, a bucchal swab, or a PAP smear. Alternatively, such a cytological sample can consist of stem cells for implantation purposes. Moreover, said multicellular sample can be a cytological sample derived from an amniocentesis.

A preferred system according to the present invention is a system wherein said molecular profiles are nucleic acid profiles. Such a nucleic acid profile can be a nucleic acid expression profile, a nucleic acid variation profile, a nucleic acid methylation profile, or other nucleic acid modification profile.

Another preferred system according to the present invention is a system wherein said molecular profiles are protein profiles. Such a protein profile is, e.g., a protein expression profile, a post-translational protein modification profile, or a protein activity profile.

Another preferred system according to the present invention is a system wherein said molecular profiles are profiles of metabolytes and other endogenous and exogenous small molecular entities (sme) including signal transduction molecules and hormones. Such a metabolite profile is, e.g., a profile of endogenous physiological or pathophysiological metabolytes or a profile of xenobiotic metabolytes (metabolytes of pharmacologically or toxicologically relevant exogenous substances).

Yet another preferred system according to the present invention is a system wherein said molecular histological analysis is performed automatically using a prediction algorithm together with said digital image and said molecular profiles.

The system of the present invention may further be configured such that input for computer aided algorithms is provided allowing an automated analysis of the modified digital images created from the multicellular sample image combined with molecular profiles of multiple molecular species to generate additional interpretive images, medical data, medical statistics, or medical reports of predictive value or diagnostic value.

Moreover, the system according to the present invention may be further configured to convert the primary data of several histological samples or cytological specimen into a computational, three-dimensional model of an organ or organism. A combination of image data of the organ, molecular signature(s) of cell(s), and phenotype of cell contained in the input sample may enable computational quantitative analysis to provide an assessment for disease status of a histological sample or cytological specimen.

Additionally, the system according to the present invention may further be configured to be capable of processing, interrogating, and/or communicating with a computer network, serving as input for a medical decision support system.

Another aspect of the present invention is a method to perform a molecular histological analysis of a multicellular sample comprising:
a) obtaining a digital image of said multicellular sample,
b) selecting a region of interest within said digital image corresponding to one cell or a number of cells within said multicellular sample,
c) isolating said cell or said number of cells selected in step b) from said multicellular sample,
d) performing a biochemical analysis to produce a molecular profile of said isolated cell or said number of isolated cells, said molecular profile comprising a plurality of molecular values, and
e) producing a modified digital image comprising said digital image obtained in step a) together with one or more of said molecular values produced in step d) to perform said molecular histological analysis.

The method according to the present invention produces modified digital images based on an original digital image of a multicellular sample (e.g., a contrast enhanced photograph using a magnification or a fluorescence image) and molecular values that are obtained by an external biochemical analysis of isolated cells. Options to isolate cells and perform biochemical analysis were already described before.

A preferred method according to the present invention is a method wherein steps b) to d) are repeated for different regions of interest.

Another preferred method according to the present invention is a method wherein steps b) to d) are repeated until all cells of the multicellular sample are analyzed.

In a preferred method according to the present invention, a plurality of modified digital images are producible, each with a different molecular value or with different groups of molecular values.

There are of course many ways to modify a digital image in order to provide support for a molecular histological analysis of a multicellular sample. Said modified digital images can help to answer questions like, e.g., where in the multicellular sample are those cells that have an overexpression of a certain gene, are there cells with a certain gene expression that have a certain distance from each other, or where are cells that have an overexpression of a certain gene while an other gene is downregulated. To answer such questions, it can be necessary to indicate one or more molecular values within the modified digital image.

In another preferred method according to the present invention, said modified digital images contain said molecular values as numerical values or indicated by color.

In yet another preferred method according to the present invention, said modified digital images comprise said digital image together with molecular values from different regions of interest.

In an also preferred method according to the present invention, said molecular values of said modified digital images are individually selected for visualization by a user.

The different alternatives to visualize different molecular values within a digital image according to user-defined criteria were already described before.

A preferred method according to the present invention is a method wherein said biochemical analysis is a gene expression analysis based on PCR amplification, preferably a real-time PCR.

As mentioned before, from the group of suitable downstream analysis processes to gain further molecular information of the cells, a PCR amplification is especially preferred because of the small amount of nucleic acid that can be obtained from only a number of isolated cells. Moreover, using a real-time PCR offers multiplexing capabilities and provides the desired amplification and the required analysis in a single process.

Another preferred method according to the present invention is a method wherein said molecular profiles are nucleic acid profiles, preferably gene expression profiles.

Using PCR as a downstream analysis process, it is possible to analyze gene expression of cells in terms of quantification of mRNA derived from individual cells isolated from a multicellular sample. Depending on the complexity of the desired molecular histological analysis, a certain number of genes need to be screened within the multicellular sample.

A more preferred method according to the present invention is a method wherein said PCR amplifies more than 5, preferably more than 10, most preferably more than 100 genes.

Note that in principle, the expression of all genes that are analyzed and summarized in the respective molecular profile can be displayed in one modified digital image using color, intensity, and/or numerical values. But since the value of such a complex digital image is questionable, it is desirable to produce a number of different modified digital images displaying only a certain amount of all analyzed genes.

Yet another preferred method according to the present invention is a method wherein said molecular profiles are protein profiles.

Alternatively to downstream processes on basis of nucleic acid analysis, it is of course possible to study the cells of the multicellular sample with respect to their protein content, resulting in a protein profile of the isolated cells.

A more preferred method according to the present invention is a method wherein said biochemical analysis is an immunoassay.

Alternatively to a process where the molecular data on protein profiles is obtained by conventional in-situ staining such as immuno-histochemistry or immuno-cytochemistry, a process can be conceived where, following (single) cell isolation, the cell content is transferred to separate reaction vessels (or surfaces) for subsequent analysis of one or more molecular properties of one or more protein species. A preferred analytical approach for this purpose is an ELISA technique or a protein expression array technique. In order to circumvent technical difficulties in multiplex-protein analysis such as limitations in sensitivity, such an analytical approach could be based, for example, on methods like immuno-PCR.

Another more preferred method according to the present invention is a method wherein said immunoassay detects more than 5, preferably more than 10, and most preferably more than 100 proteins.

Yet another aspect of the present invention is a method to identify the location of characteristic cells within a multicellular sample in order to perform a molecular histological analysis, said method comprising:
a) providing a digital image of said multicellular sample together with molecular profiles of a cell or number of cells within said multicellular sample, each molecular profile comprising a plurality of molecular values and is linked to a region of interest within said digital image corresponding to the respective cell or number of cells, and
b) highlighting in a modified digital image those cells within said multicellular sample that fulfill a defined molecular criteria, wherein said molecular profiles are obtained by isolating a cell or a number of cells from said multicellular sample.

As mentioned before, this embodiment of the present invention refers to a method that enables a user to identify characteristic cells within a multicellular sample that fulfill a certain molecular criteria by examination of a modified digital image. For this purpose, each cell of the multicellular sample corresponds to a so-called region of interest within the digital image such that a defined position or area exists for each cell in order to modify the digital image at said position or area with the respective molecular values.

In a preferred method to identify the location of characteristic cells according to the present invention. said region of interest of the respective cells is defined by two or more coordinates.

Yet another preferred method to identify the location of characteristic cells according to the present invention is a method wherein said highlighting is performed by numerical values or by color.

The alternatives to define the ROIs within the digital image as well as the subsequent isolation of the corresponding cells within the multicellular sample and the indication of molecular values within modified digital images were described in detail before.

In yet another preferred method to identify the location of characteristic cells according to the present invention, said molecular profiles are nucleic acid profiles, preferably gene expression profiles. A preferred method to identify the location of characteristic cells according to the present invention is a method wherein said molecular criteria is the expression level of a gene or the expression level of a associated group of genes.

Another preferred method to identify the location of characteristic cells according to the present invention is a method wherein said molecular profiles are protein profiles. A more preferred method to identify the location of characteristic cells according to the present invention is a method wherein said molecular criteria is the concentration of a protein.

As mentioned before, the molecular histological analysis of the multicellular sample is preferably based on the measurement of the nucleic acid or protein content of the isolated cells.

Still another aspect of the present invention is a computer program to generate a plurality of modified digital images from the digital image of a multicellular sample, said digital images being modified with one or more molecular values of the respective cell or number of cells within said multicellular sample, said computer program comprising:
means for loading said digital image of said multicellular sample and a plurality of molecular values of a cell or number of cells from said multicellular sample, each molecular value being linked to a region of interest within said digital image corresponding to the respective cell or number of cells, and
means for generating a modified digital image comprising said digital image and a number of values selected from said plurality of molecular values, wherein said molecular values are obtained by isolating a cell or a number of cells from said multicellular sample.

Preferably, said computer program generates said plurality of modified digital images in order to perform a molecular histological analysis that is based on said modified digital images.

The computer program to generate a plurality of modified digital images according to the present invention enables a user to generate modified digital images that highlight those cells within a multicellular sample that fulfill a certain user-defined molecular criteria. This modified digital image can be used, e.g., to verify histological hypotheses.

For this purpose it is essential that the computer program is able to link molecular profiles to defined regions of a digital image while loading said digital image and said molecular profile. The defined regions of a digital image in general correspond to a single cell or a group of neighboring cells, and said region is called a region of interest (ROI).

In a preferred computer program according to the present invention, said region of interest of the respective cells is defined by one or more coordinates.

In another preferred computer program according to the present invention, said selected molecular values on said modified digital images are indicated as numerical values or indicated by color.

The alternatives for defining an ROI and for indicating molecular values were already described in detail before.

In yet another preferred computer program according to the present invention, a threshold of a certain molecular value of interest is selected, and all cells are highlighted within said modified digital image that exceed or that not exceed said threshold of said molecular value of interest.

In this special embodiment of the computer program according to the present invention, the criteria that is indicated within the modified digital image is a threshold test for a certain molecular value. Such a threshold test indicates, e.g., all cells within the multicellular sample with an up or down regulation of a gene of interest, wherein the threshold is the normal expression of said gene.

Another preferred computer program according to the present invention is a computer program wherein said molecular values are nucleic acid expression values.

Yet another preferred computer program according to the present invention is a computer program, wherein said molecular values are protein expression values.

Moreover, an aspect of the present invention is a method for managing molecular histological experiments of a multicellular sample, said method comprising:
a) loading a digital image of said multicellular sample into a software application,
b) defining regions of interest within said digital image that are linked to corresponding positions of cells within said multicellular sample, and
c) loading a plurality of molecular profiles of a cell or number of cells from said multicellular sample into said software application, each molecular profile comprising a plurality of molecular values,
   wherein said software application is structured such that said plurality of molecular profiles loaded in step c) are linked to the corresponding region of interest defined in step b) within said digital image loaded in step a), and wherein said molecular profiles are obtained by isolating a cell or a number of cells from said multicellular sample.

In a preferred method for managing molecular histological experiments according to the present invention, said region of interest is defined by one or more coordinates. In another preferred method for managing molecular histological experiments according to the present, invention said molecular profiles are obtained by isolating a cell or a number of cells from said multicellular sample. In yet another preferred method for managing molecular histological experiments according to the present invention, said molecular profiles are nucleic acid profiles, preferably gene expression profiles.

A preferred method for managing molecular histological experiments according to the present invention is a method wherein said molecular profiles are protein profiles.

Another preferred method for managing molecular histological experiments according to the present invention is a method wherein a molecular histological analysis of said multicellular sample is performed.

As mentioned before, a molecular histological experiment is the analysis of a multicellular sample with respect to morphological as well as molecular aspects.

Yet another preferred method for managing molecular histological experiments according to the present invention is a method wherein a modified digital image of said multicellular sample is produced in order to perform a histological analysis of said multicellular sample.

After the molecular histological experiment is performed, the obtained molecular values are used to produce one or more modified digital images, said modified digital images being the basis for a histological analysis of said multicellular sample.

An also preferred method for managing molecular histological experiments according to the present invention is a method wherein said software application is a database, an image-based application, or a combination thereof.

A more preferred method for managing molecular histological experiments according to the present invention is a method wherein said image-based application is a TIFF application.

The different software applications that are applicable throughout the present invention were already described in detail before.

In addition, the present invention relates to a computer program to identify the location of characteristic cells within a multicellular sample in order to perform a molecular histological analysis, said computer program comprising:
a first application to load a digital image of multicellular sample, to define regions of interest within said digital image that are linked to corresponding positions of cells within said multicellular sample, and to save said digital image together with said regions of interest as a first image file,
a second application to combine said first image file with a plurality of molecular profiles comprising a plurality of molecular values and to save said first image file together with said plurality of molecular profiles as a second image file, wherein said plurality of molecular profiles are linked to the corresponding region of interest within said digital image, and
a third application to extract modified digital images from said second image file according to user-defined molecular criteria, wherein said modified digital images highlight cells that fulfill said user-defined molecular criteria, wherein said molecular profiles are obtained by isolating a cell or a number of cells from said multicellular sample.

Details of the three applications that are part of the computer program to identify the location of characteristic cells according to the present invention were already described in detail before.

In brief, the first application is an imaging application to load a digital image, to define ROIs within said digital image, and to save the digital image together with defined ROIs as a first image file. The second application is able to add to said first image file a plurality of molecular profiles and to save both components together as a second image file. The third application is necessary to extract modified digital images from the second image file.

A preferred computer program to identify the location of characteristic cells according to the present invention is a program wherein said region of interest is defined by one or more coordinates.

Another preferred computer program to identify the location of characteristic cells according to the present invention is a program wherein said molecular profiles are nucleic acid profiles, preferably gene expression profiles.

Yet another preferred computer program to identify the location of characteristic cells according to the present invention is a program wherein said molecular profiles are protein profiles.

In a preferred computer program to identify the location of characteristic cells according to the present invention, said modified digital image highlights cells by color.

In another preferred computer program to identify the location of characteristic cells according to the present invention, said computer program is an image-based application, preferably a TIFF application.

The present invention also relates to a database for managing molecular histological experiments of a multicellular sample, said database comprising:
a cell table comprising cell numbers together with the corresponding position within said multicellular sample,
a molecular value table comprising molecular profiles of cells within said multicellular sample, each molecular profile comprising a plurality of molecular values, and
a digital image table comprising digital images of said multicellular sample and coordinates of cells within said digital image,
   wherein said cell table is linked with said molecular value table and with said digital image table such that each cell of said multicellular sample is linked to the corresponding coordinates within the respective digital image and to the corresponding molecular profile, wherein said database is designed such that a modified digital image based on a digital image from said digital image table highlighting cells fulfilling a plurality of user-defined molecular criteria is producible, and wherein said molecular profiles are obtained by isolating a cell or a number of cells from said multicellular sample.

Details of the database components to managing molecular histological experiments of a multicellular sample according to the present invention were already described in detail before.

In brief, the database according to this embodiment of the present invention comprises a cell table summarizing information about each cell of interest that is used for the molecular histological experiment, a molecular value table comprising all molecular values obtained by the biochemical analysis, and a digital image table storing digital images of the multicellular sample together with coordinates that define the ROIs.

In a preferred database according to the present invention, said molecular profiles are nucleic acid profiles, preferably gene expression profiles. In another preferred database according to the present invention, said molecular profiles are protein profiles. In yet another preferred database according to the present invention, said modified digital image highlights said cells by color.

The figures are provided to aid the understanding of the present invention, the true scope of which is set forth in the appended claims. It is understood that modifications can be made in the procedures set forth without departing from the spirit of the invention.

What is claimed is:

1. A system to perform a molecular histological analysis of a multicellular sample, the system comprising:
an optical device to produce a digital image of said multicellular sample,
a computer means to select a region of interest within said digital image corresponding to one cell or a number of cells within said multicellular sample,
an isolation device to isolate said cell or said number of cells,
an analysis device to perform a biochemical analysis and to produce a molecular profile of said isolated cell or number of cells, said molecular profile comprising a plurality of molecular values,
a software application to store said digital image and said molecular profile in such a way that said molecular profile is linked to the region of interest within said digital image, and
a visualizing means to present a modified digital image comprising said digital image produced by the optical device together with one or more of said molecular values, wherein said molecular histological analysis is based on said modified digital image.

2. The system of claim 1 wherein said optical device is a fluorescence microscope.

3. The system of claim 1 wherein said isolation device is a laser assisted microdissection device.

4. The system of claim 1 wherein said number of isolated cells is selected from the group consisting of less than 100 cells, less than 10 cells, and less than 5 cells.

5. The system of claim 1 wherein said analysis device is selected from the group consisting of an immunoassay device, a mass spectrometer device, a nucleic acid sequencing device, a nucleic acid hybridization assay device, a surface plasmon resonance device, a gel electrophoresis device, and a PCR device.

6. The system of claim 1 wherein said software application comprises an application selected from the group consisting of database applications, image-based applications, and combinations thereof.

7. The system of claim 1 wherein said multicellular sample is selected from the group consisting of a tissue sample, a cell culture sample, a biopsy sample, and a cytological sample.

8. The system of claim 1 wherein said molecular profiles are nucleic acid profiles.

9. The system of claim 1 wherein said molecular profiles are protein profiles.

10. The system of claim 1 wherein said molecular histological analysis is performed automatically using a prediction algorithm together with said digital image and said molecular profiles.

11. A method to perform a molecular histological analysis of a multicellular sample, comprising:
obtaining a digital image of said multicellular sample,
selecting a region of interest within said digital image corresponding to one cell or a number of cells within said multicellular sample,
isolating said cell or cells from said multicellular sample,
performing a biochemical analysis to produce a molecular profile of said isolated cell or cells, said molecular profile comprising a plurality of molecular values, and
producing a modified digital image comprising said digital image of said multicellular sample together with one or more of said molecular values, and performing said molecular histological analysis using said modified digital image.

12. The method of claim 11 wherein said biochemical analysis is a gene expression analysis comprising PCR amplification.

13. The method of claim 11 wherein said biochemical analysis comprises an immunoassay.

14. A method to identify the location of a characteristic cell or number of cells within a multicellular sample in order to perform a molecular histological analysis, said method comprising:
providing a digital image of said multicellular sample together with a molecular profile of said cell or number of cells, said molecular profile comprising a plurality of molecular values and linked to a region of interest within said digital image corresponding to the cell or number of cells,
highlighting in a modified digital image those cells within said multicellular sample that fulfill a defined molecular criteria,
wherein said molecular profiles are obtained by isolating a cell or a number of cells from said multicellular sample.

15. A computer program to generate a plurality of modified digital images from a digital image of a multicellular sample, said digital images being modified with one or more molecular values of a cell or number of cells within said multicellular sample, said computer program comprising:
loading said digital image of said multicellular sample and a plurality of molecular values of cells or number of cells from said multicellular sample, each molecular value is linked to a region of interest within said digital image corresponding to the respective cell or number of cells, and
generating a modified digital image comprising said digital image and a number of selected molecular values,
wherein said molecular values are obtained by isolating a cell or a number of cells from said multicellular sample.

16. A method for managing molecular histological experiments of a multicellular sample, said method comprising:
loading a digital image of said multicellular sample into a software application,
defining a region of interest within said digital image that is to a corresponding position of a cell number of cells within said multicellular sample, and
loading a plurality of molecular profiles of said cell or number of cells from said multicellular sample into said software application, each molecular profile comprising a plurality of molecular values,
wherein said software application is structured such that said plurality of molecular profiles are linked to the corresponding region of interest within said digital image and
wherein said molecular profiles are obtained by isolating a cell or a number of cells from said multicellular sample.

17. The method of claim 16 wherein a modified digital image of said multicellular sample is produced in order to perform a histological analysis of said multicellular sample.

18. The method of claim 16 wherein said software application is selected from the group consisting of database applications, image-based applications, and combinations thereof.

19. A computer program to identify the location of characteristic cells within a multicellular sample in order to perform a molecular histological analysis, said computer program comprising:
a first application which loads a digital image of multicellular sample, defines regions of interest within said digital image that are linked to corresponding positions of cells within said multicellular sample, and saves said digital image together with said regions of interest as a first image file,
a second application which combines said first image file with a plurality of molecular profiles comprising a plurality of molecular values and saves said first image file together with said plurality of molecular profiles as a second image file, wherein said plurality of molecular profiles are linked to corresponding regions of interest within said digital image, and wherein said molecular profiles are obtained by isolating a cell or a number of cells from said multicellular sample, and
a third application which extracts modified digital images from said second image file according to user-defined molecular criteria, wherein said modified digital images identifies the location of the characteristic cells by highlighting cells that fulfill said user-defined molecular criteria.

20. The computer program of claim 19 wherein said computer program comprises a TIFF application.

21. A database for managing molecular histological experiments of a multicellular sample, said database comprising:
a cell table comprising multiple cell numbers and corresponding positions within said multicellular sample,
a molecular value table comprising molecular profiles of cells within said multicellular sample, each molecular profile comprising a plurality of molecular values, and
a digital image table comprising digital images of said multicellular sample and coordinates of cells within said digital image,
wherein said cell table is linked with said molecular value table and with said digital image table such that each cell of said multicellular sample is linked to the corresponding coordinates within the respective digital image and to the corresponding molecular profile,
wherein said database is designed such that a modified digital image based on a digital image from said digital image table highlighting cells fulfilling a plurality of user-defined molecular criteria is producible, and
wherein said molecular profiles are obtained by isolating a cell or a number of cells from said multicellular sample.
